(19) United States
(12) Patent Application Publication
(10) Pub. No.: US 2008/0166806 A1
(43) Pub. Date: Jul. 10, 2008

(54) Attenuated Effector Ligand

(76) Inventor: Tom Wehrman, Fremont, CA (US)

Correspondence Address: Mark Wehrman, 1310 Holly Creek Ln, Chapel Hill, NC, 27516, US

(21) Appl. No.: 11/621,103

(22) Filed: Jan. 8, 2007

(51) Int. Cl. C12N 5/00

(52) U.S. Cl. 435/375

(57) ABSTRACT

Methods and compositions are provided for effecting physiological processes using a conjugate of a binding member binding to a surface membrane marker and a ligand for a cell surface receptor, where the ligand is attenuated as compared to the natural ligand. The composition finds particular application in discriminating between cells that share the receptor and can be distinguished by the surface membrane marker.

### FIELD OF THE INVENTION

The field of the invention is modulating cell physiology.

### BACKGROUND

In many contexts one is interested in modifying the physiology of cells, for example, abundance of specific cell types in a cellular population. There are both in vitro and in vivo applications. For example, one may be interested in removing a particular group of cells from a cell population to provide a pure cell population. When using cells in research, one may be interested in removing cells at a particular level of differentiation, so as to have a population at a desired level of differentiation. Alternatively, one may wish to activate cells at a particular location in a host. Other examples will come to mind.

More importantly, in a mammalian host there will be numerous instances when one wishes to encourage or discourage cellular expansion, activation or inactivation. In many situations one may wish to expand or diminish a particular lymphocyte population, such as in the case of inflammation, infectious diseases and cancer. Where there is a diseased cellular population, such as cancer cells, one would wish to destroy the cancer cells. In the regeneration process, one may wish to activate hematopoietic cells at the location of the injury. However, for many of the treatments there is no discrimination between target cells and other cell having the same receptor, such as healthy cells and cancerous cells. In cancer, many of the chemotherapeutic agents kill any rapidly expanding cell population, such as the hematopoietic cells, leaving the host immunocompromised. In other situations, one may wish to reduce a population of activated cells, as compared to quiescent cells. For example, with diabetics, one would wish to control hematopoietic cells that are specific for islet cells, rather than an overall diminution of the immune cells. There is, therefore, a need to be able to distinguish between a cell population one wishes to control and a cell population that is to be minimally affected.

### SUMMARY OF THE INVENTION

Methods and compositions are provided for differential modulation of cell physiology using a conjugate comprising a binding entity specific for a target cell population joined to an attenuated ligand. Each attenuated ligand has a substantially lower capability for affecting the activity of its complementary receptor, usually having an affinity for its receptor substantially lower than the wild-type ligand. The ligand binds to the target complementary cellular receptor at a much higher localized concentration as a result of the binding entity binding to a cellular marker and modulates the physiology of the cell to which it binds. By virtue of the attenuated ligand, one is able to localize the cellular effect.

### DESCRIPTION OF THE SPECIFIC EMBODIMENTS

Use of conjugates comprising a binding entity specific for a marker associated with a target cell population joined to an attenuated ligand for differential modulation of cell physiology of the target cell population, e.g. population growth or diminution, is provided. The target cell population is a member of a mixed cell or heterogeneous cell population that may be a single type of cell that is in different states, e.g. neoplastic, activated, etc., or two different types of cells that are found in proximity in the host. The mixed cell population is combined with the conjugate whereby the receptor-containing target cell physiology is modified. The binding entity will bind to a surface membrane protein marker that is relatively specific for the marker population, while the attenuated ligand will bind to all cells comprising the receptor for the ligand. The binding of the binding entity to the marker-containing-cells results in a differential effect on cells comprising the receptor. The surface membrane marker may be on the target cells or other cells, where the other cells will be localized or found in proximity to the target cells. The effect of the conjugate on the target cells may be related to growth, cell death, cell proliferation or cell quiescence, or to some other physiological effect, activation, inactivation, translocation, extravasation, etc. For the most part, the subject invention is concerned with mammalian hosts, but may also be used in vitro or with other hosts in vivo.

The subject invention is directed to the situation where one can distinguish in a population of cells sharing the same receptor, a target population differing in a property of interest from the other cells sharing the same receptor. Two ways are selected for identifying the target population. The first way is by the target population having a higher level of a surface marker than the other cells sharing the same receptor. The second way is where the target population is in proximity to a first cell population. The first cell population differs from a second cell population sharing the same receptor in that the first cell population is in proximity to the target cells. As is well known, organs have a complex mixture of cell types and may have cells that infiltrate the organ. As an illustration, hematopoietic cells can infiltrate tissue causing inflammation. One can then use the presence of markers on the inflamed tissue to localize the subject conjugate to the site of inflammation, while the ligand binds to the hematopoietic cells. In effect, one acts only on those hematopoietic cells that are associated with the tissue, particularly the inflamed tissue where inflammation results in a specific marker, rather than all hematopoietic cells in the host.

There are many situations where cells are distinguished by responding to an exogenous, endogenous or external stimulus. Inflammation is an example. Cells respond to various hormones, autocrine, exocrine or endocrine, that can result in a change in populations of surface cell markers or their migration to a localized site in a host.

By virtue of being able to distinguish the target cells from other cells sharing the same receptor, one provides a higher localized concentration of the attenuated ligand, where the localized concentration offsets the attenuated activity and provides for at least a substantial portion of the wild-type ligand activity. That is, it is believed that by having a much higher localized concentration of the ligand at the cell surface, even though the affinity of the ligand may be lower than the wild-type ligand, there will be a sufficient level of binding of the attenuated ligand to the complementary receptor to transduce a signal into the cell or provide the desired inhibition of signal transduction. In referring to wild-type it is intended to include natural compounds that can be modified or mutated to reduce their affinity to a naturally occurring cell surface membrane receptor and to unnatural or synthetic compounds that do not come with the binding range affinity of drugs, that is, they have a binding affinity of less that 1 μM, usually less that 50 nM.

The conjugate is comprised of the binding entity and the attenuated ligand. The binding entity is any compound that binds to the marker protein with an affinity of at least about 50 nM, generally at least about 10 nM, preferably at least about 1 nM. The binding entity may be a polypeptide, a natural product, a synthetic organic molecule, or the like. Usually, the binding entity will be at least about 200 Dal and not more than about 800 kDal, usually being at least about 250 Dal and not more than about 200 kDal. Conveniently, the binding entity may be an antibody or binding modification thereof, e.g. scFv, Fab, F(ab)2, etc., generally being an IgG antibody. The antibody may be naturally derived from a host, hybridoma, etc., or may be modified, e.g. humanized. Besides antibodies, other binding entities include lectins, ligands, such as drugs, hormones, polypeptides, etc. and unnatural binding entities, such as aptamers.

The binding entity will bind to a surface membrane protein as the surface marker. The surface membrane protein will allow discrimination as to a particular population of cells. The cells may be diseased cells, e.g. cancer cells, precancerous cells, infected cells, cell comprising at least one mutated protein, abnormal cells, etc., or normal cells, e.g. activated cells, proliferating cells, quiescent cells, etc.

The attenuated ligand may be any composition that has the following characteristics: it binds to the target receptor with less than about 10% of the binding affinity of the wild-type ligand, usually less than about 1%, and may be as low as less than about 0.1%; and binds to a receptor that is in at least one pathway that affects cell physiology; or activation by the attenuated ligand requires a substantially higher number. Depending upon the particular context, the conjugate may bind to a target cell marker or a marker on a different cell, where one is interested in modifying the target cell that is in the vicinity of a different cell that allows for discrimination of the target cell from other cells having the same receptor. The attenuated ligand retains the capability of activating the receptor and may be a mutated wild-type ligand, a naturally occurring compound that binds to the receptor, or a synthetic organic molecule that binds to the receptor. By having a large fraction of the total number of receptors being activated by virtue of the high localized concentration of the attenuated ligand, one can obtain an effect analogous to the wild-type ligand binding.

In effect, at its concentration in a medium, e.g. blood, the attenuated ligand, by itself, is substantially less effective than the wild-type ligand in modulating the activity of the receptor on a cell due to the low level of binding to receptors on the cell. In effect, one should be able to give at least 5 times the amount of wild-type ligand However, when the attenuated ligand is bound to the binding entity with the concomitant increase in local concentration of the attenuated ligand, the activity of the attenuated ligand can approach that of the wild-type ligand in modulating the activity of the receptor, restoring at least about 1%, desirably at least about 5%, and up to 90% or more, of the activity of the wild-type ligand at saturation, usually maximum physiologic response, in producing a cellular physiological response.

Depending upon the nature of the binding entity and the attenuated ligand there will be a wide variety of methods for preparing the conjugate. Where both are polypeptides, one may use genetic engineering to produce a gene that encodes for the conjugate. Methods for joining sequences encoding for polypeptides are well known in the art and need not be exemplified here. See, for example, Molecular Cloning: A Laboratory Manual, Sambrook and Russell, 2001. Alternatively, one may use chemical conjugation between polypeptides, where the polypeptides are synthesized and provided with active groups for covalent linking, such as cysteines, maleimides for bonding to a cysteine, etc., or using natural sequences for covalent bonding, such as transamination. Where the attenuated ligand is a non-protein organic molecule, there are numerous functionalities that can be used to randomly or specifically bind to the functional groups of a protein, such as sulfhydryl, hydroxyl, carboxyl, or amino. See, for example, U.S. Patent application nos. 2004/0067537; 2005/0003431; and 2005/0287518 and references cited therein; and U.S. Pat. Nos. 4,487,715 and 6,054,127, and references cited therein, the appropriate portions thereof showing protein conjugation incorporated herein by reference.

The subject method may be used in vitro. There are many situations where a cell population is mixed with a different cell population. For example, one may wish to remove a particular cell population while retaining the remaining population. This can involve the presence of activated and quiescent cells, cancerous and normal cells, or other populations that have distinguishing surface targets. By combining the mixture with the subject conjugate, where binding of the attenuated ligand results in cell death, the undesired population may be substantially removed from the desired population. This can find application where one wishes to determine the level of expression of one or more proteins in the desired population, where the undesired population would confuse the amount. Alternatively, one may interested in stage of maturation of cells from precursor cells, where one could remove the cells at a higher or lower stage of maturation.

The method may be used in vivo. The method finds use for research as a prophylactic or as a therapeutic. As indicated previously, the attenuated ligand will provide the effect of the wild-type ligand, resulting in activating cell pathways that result in cell proliferation, cell differentiation or maturation, cell migration and cell death. In performing research, the ability to remove a target cell population in vivo allows one to study the effect of the absence of the target population. During development of a fetus, one can introduce the subject conjugate during a specific period of development and determine the effect of the reduction or loss of a cell population on the fetal development. Similarly, one can enhance the proliferation or differentiation of cells during fetal development, allowing one to identify the effect of the target population on the fetal development. In mature hosts, one can determine the effect on the absence or presence of a particular cell population on the susceptibility to infection, response to a toxic agent, physiologic response to learning, sleep, etc.

The subject method can be used for activating immune cells at a site of infection, e.g. bacterial or viral infection. Where the infectious agent provides for a cell surface marker, a conjugate can be prepared that will have a binding entity that binds to the marker of the infectious agent and an attenuated ligand that binds to an immune cell receptor to activate the immune cell. In this way there will be a high localized concentration of the attenuated ligand on the infected cell to enhance the probability that immune cells will be primarily activated in the locale of the infected cell and not at other sites. Similarly, in the case of a tumor, one can have the binding entity specific for a tumor marker and NK cells activated by the attenuated ligand in the vicinity of the tumor.

Where there is a localized concentration of target cells, the binding entity may bind to one of the target cells, while the attenuated ligand may bind to a receptor of a different target cell. A cross-linked aggregation is envisioned where the conjugate can serve to cross-link the target cells through the binding of the binding entity to a target cell and the attenuated ligand to a different target cell and also have a conjugate bound solely to a single target cell. Where the result of ligand binding is secretion of a protein that affects the target cells, this will result in localized activity due to the presence of the secreted protein in the locale where the conjugate is bound.

In mixtures of cells one can diminish or enhance proliferation of a particular cell group. For example, where there are different alleles are different cells, by having the binding entity bind to one rather than the other allele, the ligand can effect proliferation or inhibit proliferation. In this way one can enhance the number of cells of one allele as compared to the other allele. In addition, where there is a mixture of cells of different type, e.g. wild-type and hyperplasic cells, such as cancer cells, and the cells differ by an idiotope, one can select one of the types of cells for effecting a pathway, while not inducing the pathway in the other type of cells. Other situations will also lend themselves to the use of the subject methodology.

There are a large number of proteins that act as ligands and activate or inactivate pathways. The ligands may be used in a variety of ways for research or therapeutic purposes in conjunction with the binding entity so as to observe a localized effect rather than a general effect. Each of these ligands may be used induce a physiologic response or by using a ligand that binds to a receptor serving as a cell surface marker, usually a ligand that will not interfere with the physiological effect of interest, a ligand may serve to bind to cell markers to differentiate cells that share the receptor. Normally, one would use Chemokines of interest include: 6Ckine464.1, 744.1 3-10C, 9E3; A: A ATAC ABCD-1 ABCD-2 ACT-2 ALP AMAC-1 AMCF-1 AMCF-2 AIF ANAP Angie Angie-2 ABCD; B beta-R1 Beta-Thromboglobulin BCA-1 BLC blr-1 ligand BMAC bolekine BRAK; C C10 CCF18 CCK1 CCL1 CCL2 CCL3 CCL4 CCL5 CCL6 CCL7 CCL8 CCL9 CCL10 CCL11 CCL12 CCL13 CCL14 CCL15 CCL16 CCL17 CCL18 CCL19 CCL20 CCL21 CCL22 CCL23 CCL24 CCL25 CCL26 CCL27 CCL28 Ck-beta-1 Ck-beta-4 Ck-beta-6 Ck-beta-7 Ck-beta-8 Ck-beta-8-1 Ck-beta-9 Ck-beta-10 Ck-beta-11 Ck-beta-12 Ck-beta-15 CTACK C7 cCAF CEF-4 CINC CINC-2-alpha CINC-2-beta CINC-2-beta-like CKA-3 CRG-2 CRG-10 CTAP-3 CXCL1 CXCL2 CXCL3 CXCL4 CXCL5 CXCL6 CXCL7 CXCL8 CXCL9 CXCL10 CXCL11 CXCL12 CXCL13 CXCL14 CXCL15 CXCL16 CIP-1; D DC/B-Ck DC-CK1 DCtactin DCtactin-beta dendrokine DNA binding protein DNAP; E ELC Eotaxin Eotaxin-2 eotaxin-3 ESkine Exodus-1 Exodus-2 Exodus-3 ENA-78 ENAP ENAP-alpha ENAP-beta Endothelial cell growth inhibitor Endothelial IL8; F FIC Finetaxin FDNCF FINAP Fractalkine; G G26 GDCF GDCF-2 GOS-19-1 GOS-19-2 GOS-19-3 GCF GCP-2 GRO1 GRO2 GRO3 GRO-alpha GRO-beta GRO-gamma; H H400 HC-11 HC-14 HC-21 HCC-1 HCC-2 HCC-3 HCC-4 H174 Heparin neutralizing protein Humig; I I-309 ILC ILINCK IMAC I-TAC Ifi10 IL8 IP-9 IP-10 IRH; J JE JSC; K K203 KC; K60 KEC KS1; L Lymphotactin L2G25B LAG-1 LARC LCC-1 LD78-alpha LD78-beta LD78-gamma LDCF LEC Lkn-1 LMC LAI LCF LA-PF4 LDGF LDNAP LIF LIX LUCT Lungkine LYNAP; M Manchester inhibitor MARC MCAF MCIF Mexikine MCP-1 MCP-2 MCP-3 MCP-4 MCP-5 MDC MEC MIP-1-alpha MIP-1-beta MIP-1-delta MIP-1-gamma MIP-3 MIP-3-alpha MIP-3-beta MIP-4 MIP-4-alpha MIP-5 Monotactin-1 MPIF-1 MPIF-2 MRP-1 MRP-2 Mtn-1 M119 MDGF MDNAP MDNCF Megakaryocyte-stimulatory-factor MGSA MGSA-alpha MGSA-beta Mig MIP-2 MIP-2-alpha MIP-2-beta MIP-2-gamma mob-1 MOC MONAP; N NC28 NCC-1 NCC-2 NCC-3 NCC-4 N51 NAF NAP-1 NAP-2 NAP-3 NAP-4 NAPS NCF NCP NJAC Neurotactin; 0 Oncostatin A; P P16 P500 PARC pAT464 pAT744 PESKY PBP PBP-like PBSF PF4 PF4-ALT PF4-ALT PF4V1 PLF PPBP; R RANTES Regakine-1; S SCM-1-alpha SCYC1 SCYC2 SCI SCYA1 SCYA2 SCYA3 SCYA4 SCYA5 SCYA6 SCYA7 SCYA8 SCYA9 SCYA10 SCYA11 SCYA12 SCYA13 SCYA14 SCYA15 SCYA16 SCYA17 SCYA19 SCYA20 SCYA21 SCYA22 SCYA23 SCYA24 SCYA25 SCYA26 SCYA27 SCYA28 SIS-alpha SIS-beta SIS-delta SIS-epsilon SIS-gamma skinkine SLC SMC-CF ST38 STCP-1 SCYB1 SCYB2 SCYB3 SCYB4 SCYB5 SCYB6 SCYB7 SCYB8 SCYB9 SCYB9B SCYB10 SCYB11 SCYB12 SCYB13 SCYB14 SCYB15 SCYB16 SDF-1-alpha SDF-1-beta SR-PSOX SCYD1 SR-PSOX; T TARC TCA-3 TCA-4 TDCF TECK TMkine TSC-1 TSG-8 TY5 TCF TCK-1 TLSF-alpha TLSF-beta TPAR-1 TSG-1 trout chemokine 2; W WECHE.

This alphabetical list of chemokines groups them into their respective families. Many of these factors have been described under different names and thus may appear several times. Chemokine receptors include: CXCR1, CXCR2, CXCR3, CXCR4, CXCR5, CXCR6 CCR1, CCR2, CCR3, CCR4, CCR5, CCR6, CCR7, CCR8, CCR9, CCR10, CCR10A, CCR10B, CCR11.

The following is a list of cytokine families of interest: Epidermal Growth Factor (EGF), Platelet-Derived Growth Factors, Fibroblast Growth Factors (FGFs), Transforming Growth Factor beta superfamily (including TGF-beta-1, TGF-beta-2, TGF-beta-3, TGF-beta-4, TGF-beta-5, Inhibins, Activin A, MIS, Bone morphogenetic proteins, MNSF) VEGF, Erythropoietin (Epo), Insulin-Like Growth Factors, Interleukins (including IL1, IL2, IL3, IL4, IL5, IL6, IL7, IL8, IL9, IL10, IL11, IL12, IL13, IL14, IL15(IL-T), IL16, IL17, IL17B, IL17C, IL17E, IL17F, IL18, IL19, IL20, IL21, IL22, IL23, IL24, IL25, IL26, IL27, IL28A, IL28B, IL29, IL30), Tumor Necrosis Factor (including TNF-alpha, TNF-beta, lymphotoxin beta, CD27 ligand, CD30 ligand, CD95 ligand, 4 1BB, Ox40 ligand, TRAIL), Interferons (including IFN-alpha, beta, gamma, delta, omega, tau, and related factors such as Limitin), Colony Stimulating Factors (CSFs: G-CSF, GM-CSF, MEG-CSA, Epo), integrins, hedgehog, IGF, Wnts, angiopoietins, Eph, Ephrins, semaphorins, neuropilins, cell adhesion molecules (CAMS), lectins, chemokines, alpha-2 macroglobulin, prostaglandins, thymic hormones, peptide hormones, c-kit ligand, Fc receptor ligands, hematopoietins (neuropoietins), lymphokines, TLR ligands, GPCR ligands, LIF, and the like.

Illustrative of the use of attenuated ligands is with activated T-cells that have specific markers as compared to other T-cells. For autoimmune diseases, inflammation or other aberrant condition, it is desirable to reduce the T-cell population involved with the aberrant condition. By employing a conjugate comprising a binding entity for CD69 and an apoptotic activating attenuated ligand, such as an attenuated FasL, TNF-α or TRAIL, one can substantially reduce the activated T-cell population without reducing the remaining T-cell population. For attenuated apoptotic inducing ligands, see for example, Schneider, et al, 1997 J. Biol. Chem. 272,18827-33 (FasL); and Van Ostade, et al., 1991 The EMBO Journal 10, 827-36 (TNF-α). These references illustrate the use of PCR for site specific mutation and mutagenesis for mutation in the encoding gene and are incorporated herein by reference. In these manners the attenuated proteins may be produced by modification of the gene encoding the protein in a directed or random manner. The resulting mutated proteins may then be screened for binding affinity and activity with cells to evaluate their activity in relation to the wild-type protein. For fusion proteins, the mutated proteins may be expressed as fusion proteins and screened in an analogous manner using cells presenting the complementary receptor and analyzing for the effect of ligand binding to the receptor. Conveniently, dosage responses can be obtained with the conjugate, the wild-type ligand and the attenuated ligand to compare their activity and determine the effect of administering the attenuated ligand on cells other than the target cells. The cells used for screening may be modified to provide for an easily observable signal upon activation or inactivation of the receptor.

The various biological outcomes of ligand binding include modulation of cell number, such as apoptosis, inhibition of cell proliferation, and proliferation, anergy, differentiation, maturation, activation, cell migration, adherence, secretion, cell-cell binding, vesicle formation, endocytosis, exocytosis, invagination, filopodia or other membrane extension, etc. Intracellular effects may include translocation, degradation, expression, exocytosis, formation of lipid rafts and caveolar pits, up regulation or down regulation of stress factors, etc. Similarly, one can treat various cancers by using a binding entity specific for cancer cells. For breast cancer, there is erb-2; colon cancer, there is EGFR; prostate cancer, there is membrane PSA; small cell lung cancer, there is EGFR; lung cancer, there is MAGE-B2 (Mizukami, et al., 2005 Cancer Sci 96, 882-8); pancreatic cancer, there is CK20 (Matos, et al., 2005 Cancer, Dec. 16); etc. By using conjugates having an antibody or modified form thereof specific for a cancer marker conjugated to an attenuated apoptotic inducing ligand, the cancer cells can be selectively killed with substantially reduced effect on other cells.

Alternatively, rather than use apoptosis, one may provide for proliferation of cancer killing cells, such as T-cells or NK cells, where the activation is in proximity to the tumor. Attenuated cytokines can find use that activate T-cells, e.g. interleukins, such as IL-2, 4, 6, 10, or other interleukins, interferons, α, β, and γ, chemokines, RANTES, CCL-10, MIP-1α, etc., growth factors, VEGF, PDGF, EGF, etc., and the like. Methods for joining the attenuated activating proteins to antibodies may be found in U.S. Pat. Nos. 6,403,769, 6,669,938 and 6,750,329, which are specifically incorporated herein by reference as to the methodology.

Conditions of interest include cancers, such as lung cancer, prostate cancer, breast cancer, colon cancer, liver cancer, lymphomas, sarcomas, skin cancer, etc. Autoimmune diseases include diabetes, Lupus erythematosis, allergies, asthma, arthritis, etc. Heart diseases include strokes, atherosclerosis, heart muscle damage, embolisms, etc. Brain diseases include diseases associated with plaque and fibrillary tangles, e.g. Alzheimers disease, ALS, etc, Parkinson's disease, senile dementia, schizophrenia, bipolar, depression, drug addiction, etc. Infectious diseases such as HIV, hepatitis, influenza, tuberculosis, helicobacter, tetanus, etc. Other conditions associated with diseases include angiogenesis, cell migration, diapedesis, capillary leakage, etc.

Methods of treatment involve administering to the host an effective amount of the subject conjugate in accordance with a predetermined regimen. Hosts include mammalian hosts, particularly primates, more particularly humans, research animals, e.g. rodentiae, primates other than humans, ovine, bovine, canine, feline, etc., domestic animals, e.g. bovine, canine, equine, lagomorpha, ovine, porcine, etc. The method of administration may be injection, intravenous, transdermal, eyedrops, eardrops, inhalants, intraperitoneal, intramuscular, and the like. Where the conjugate is other than a protein and even with some proteins, administration may be oral or parenteral.

The subject conjugates may be used with other compositions for a particular purpose. For example, for the treatment of cancer, the subject compositions may be used in conjunction with known chemotherapeutics at their conventional dosage or diminished dosage. Similarly for the treatment of inflammatory diseases, known anti-inflammatory agents may be used, e.g. NSAIDs. In the case of inhibiting angiogenesis, known antiangiogenesis agents may be used. The particular formulation will depend upon the nature of the conjugate, the nature of the treatment, the disease or aberrant condition, the site of the condition, the frequency of administration, the effective dosage, side effects and the like.

A conjugate useful in the present invention can be formulated as a pharmaceutical composition. Such a composition can then be administered parenterally in dosage unit formulations containing conventional nontoxic pharmaceutically acceptable carriers and vehicles as desired. The term parenteral as used herein includes subcutaneous administration, intravenous administration, or local infusion techniques. Formulation of drugs is discussed in, for example, Hoover, John E. (ed.), Remington's Pharmaceutical Sciences (18th Edition), Mack Publishing Co., Easton, Pa., 1990 and Liberman, H. A. and Lachman, L., Eds., Pharmaceutical Dosage Forms, Marcel Decker, New York, N.Y., 1980.

Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful.

For therapeutic purposes, formulations for parenteral administration can be in the form of aqueous or non-aqueous isotonic sterile injection solutions or suspensions. These solutions and suspensions can be prepared from sterile powders or granules having one or more of the common carriers or diluents well known in the art. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

A compound useful in the present invention can also be formulated into liposomes, as discussed in Hoover, John E. (ed.), Remington's Pharmaceutical Sciences (18th Edition), Mack Publishing Co., Easton, Pa., 1990, p. 1691. Liposomes are formed by dispersing phospholipids in an aqueous medium. Water- or lipid-soluble substances such as a peptide of the invention can be entrapped in the aqueous space within a liposome, or within the lipid bilayers of the liposome, respectively.

The following examples are offered by way of illustration and not by way of limitation.

### EXPERIMENTAL

### Example 1 Burkitt's Lymphoma

The CD20 antigen is highly expressed on B-cells including Burkitt's lymphoma. Further, FasL treatment has been shown to cause apoptosis of various cell types including those derived from multiple myeloma (MM), acute myeloid leukaemia (AML), acute lymphoblastic leukaemia (ALL) and Burkitt's lymphoma.

Mouse monoclonal anti-CD20 is purchased from any commercial vendor. Following the procedure of Schneider, et al., supra, the mutated FasL (P206R) is produced. The mutated FasL ligand is purified and conjugated to the antibody with SMCC in accordance with known procedures. The resulting protein conjugate is then ready to be used.

To demonstrate the effectiveness of the conjugate as compared to the wild-type and mutated FasL, 104 Burkitt lymphoma cells in 100 μl are placed in each of the wells of a 96 well plate at a density of 105/ml. Dose response over a range of 1 μM to 1 nM is determined for each of the ligands. It is found that the rank order of effectiveness is wild-type, conjugate and mutated FasL.

### Example 2

To design a system that utilizes the ADEL principle, we first mutated a DNA sequence encoding the wt TNF-alpha ligand at the position that corresponds to position 84. Mutation of this amino acid residue was previously shown to dramatically decrease the affinity of the ligand for the TNF receptor. Thirteen point mutants were made. The DNA encoding these mutant proteins was then cloned into a bacterial expression plasmid. The plasmid was introduced into E. coli and the resulting bacteria were induced to express the protein in the cytosol.

The mutant TNF-alpha ligands were purified from the bacteria and tested for their activity on mammalian cells. Activation of the TNF receptors on mammalian cells results in degradation of I kappa B. Thus measuring IKB levels is used as an indication of TNFR activation, and indirectly, the activity of the mutant ligands. We measured IKB activity in Hela cells in response to varying concentrations of the wt and mutant purified ligands. We found several ligands that showed decreased activity over a 10,000 fold range in comparison to the wt ligand.

To test whether localizing the ligands to the cellular surface would cause an increase in their activity by artificially increasing their concentration in proximity to their target receptors we conjugated the purified ligands to antibodies specific for a cell surface protein on the target cells, and a control antibody that does not bind the target cells. Addition of the control antibody-mutant ligand conjugates induced TNFR activity at the same concentrations as the purified ligands that were not conjugated to an antibody. Thus the conjugation to this antibody neither increased nor decreased the ability of the mutants to activate the TNFR. However the ligand-antibody complexes that bind the target cells via the antibody moiety were able to activate the TNFRs on the target cells at much lower concentrations than the ligands that were provided freely in solution or conjugated to the control antibody.

For peptides Angio 1 to Angio 4: A solution of 5 ml of 2 mg/ml Q.beta. capsid protein in 20 mM Hepes. 150 mM NaCl pH 7.4 was reacted for 30 minutes with 507 .mu.l of a solution of 13 mg/ml Sulfo-MBS (Pierce) in H.sub.20 at 25.degree. C. on a rocking shaker. The reaction solution was subsequently dialyzed twice for 2 hours against 2 L of 20 mM Hepes, 150 mM NaCl, pH 7.4 at 4.degree. C. 665 .mu.l of the dialyzed reaction mixture was then reacted with 2.8.mu.l of each of the corresponding 100 mM peptide stock solution (in DMSO) for two hours at 25.degree. C. on a rocking shaker. The reaction mixture was subsequently dialyzed 2.times.2 hours against 2 liters of 20 mM Hepes, 150 mM NaCl, pH 7.4 at 4.degree. C.

For peptides Angio 5-9 and Angio 13-15: A solution of 3 ml of 2 mg/ml Q.beta. capsid protein in 20 mM Hepes. 150 mM NaCl pH 7.2 was reacted for 50 minutes with 86.mu.l of a solution of 100 mM SMPH (succinimidyl-6-(.beta.-maleimidopropionoamido hexanoate, Pierce) in DMSO at 25.degree. C. on a rocking shaker. The reaction solution was subsequently dialyzed twice for 2 hours against 2 L of 20 mM Hepes, 150 mM NaCl, pH 7.2 at 4.degree. C. 514 .mu.l of the dialyzed reaction mixture was then reacted with 3.6 .mu.l of each of the corresponding 100 mM peptide stock solution (in DMSO) for 4 hours at 25.degree. C. on a rocking shaker. The reaction mixture was subsequently dialyzed 2.times.2 hours against 2 liters of 20 mM Hepes, 150 mM NaCl, pH 7.2 at 4.degree. C.

A solution of each of 2 mg/ml TNF-α (mutated at A84 to 1, N, P or R)(5 ml) in 20 mM Hepes, 150 mM NaCl pH 7.4 is reacted for 30 minutes with 0.5 ml of a solution of 15 mg/ml of Sulfo-MBS (PierceP in water at 25° C. on a rocking shaker. After dialyzing twice for 2 h against 2 L of 20 mM Hepes, 150 mM NaCl pH 7.4 at 4° C., 750 μl of the dialyzed reaction mixture is reacted with 10 μl of anti-ERB-2 modified with approximately 1 thioglycolic acid per antibody.

β-gal was purchased from Sigma. Bio-Gel P-30 size exclusion resin and Bio-Spin disposable columns were from Bio-Rad. Succinimidyl-4-(N-maleimidomethyl)cyclohexane-1-carboxylate (SMCC) was obtained from Pierce. CD4 antibody (clone RM4-5) was kindly provided by BD Pharmingen. β-gal was resuspended in PBS at 10 mg/ml and purified on a P-30 column equilibrated in PBS to remove residual Tris salts. SMCC (100 equivalents) was added and the reaction was allowed to proceed for 1 hour. The reaction was then exchanged into “coupling buffer” (50 mM MES, 2 mM EDTA, pH 6.0) on a P-30 column. Concurrently, antibody (0.5 mg/ml) was reduced with 20 mM dithiothreitol for 30 min, then exchanged into coupling buffer on a P-30 column. The reduced antibody was added to the β-gal-SMCC in a 2-6 fold molar excess, and the coupling was allowed to proceed for 1 hour. The reaction was quenched by addition of 100 equivalents of β-mercaptoethanol for 30 min. After pelleting to remove precipitated products, the reaction was purified on a P-30 column equilibrated in PBS to obtain CD4-β-Gal. The CD4-β-gal antibody and control β-gal labeled antibody were each assayed for β-gal activity in vitro and amounts of antibody with equivalent enzyme activities were injected intravenously.

Each of FGF (mutated at A84 to 1, N, P or R) is suspended in PBS at 10 mg/ml and purified on a P-30 column. SMCC(Bio-Rad. Succinimidyl-4-(N-maleimidomethyl)cyclohexane-1-carboxylate)(100 equivalents) is added and the reaction allowed to proceed for 1 h. The reaction mixture is then exchanged into coupling buffer (50 mM MES, 2 mM EDTA, pH 6.0) on a P-30 column. Concurrently, anti-CD-4 is reduced with 20 mM dithiothreitol for 30 min, then exchanged into coupling buffer on a P-30 column. The reduced antibody is added to each of the mutated TNF-α in a 2-6 molar excess and the reaction allowed to proceed for 1 h. The reaction is quenched by addition of 100 equivalents of β-mercaptoethanol for 30 min. After pelleting to remove precipitated material, the reaction is purified on a P-30 column equilibrated in PBS to obtain the antibody TNF-α conjugate. The product is then used directly.

A cell culture derived from 0.5 ml of mouse peripheral blood is obtained by tail vein bleed into an anticoagulant and the blood centrifuged and the supernatant is recovered. The cells are then plated in RPMI 10% inactivated FBS. To 105 cells is added 1 μg of the conjugate prepared above and the cell culture incubated under conventional conditions for 48 h. The cell culture is then analyzed for CD-4+ cells. As compared to a control culture there is a substantially fewer number of CD-4+ cells.

It is evident from the above results that the subject invention provides an effective method for removing undesired cell populations from a mixture of cells. One can select for cells that can be differentiated by a cell marker from other cells having the same receptor, where the cells can be removed without physical separation. In addition, the subject compositions can be used for treating various conditions where one does not wish to physiologically affect cells having the same receptor as the target cell.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for differentially modulating the physiological function of cells having a predetermined receptor in a mixture of cells comprising cells sharing the same predetermined receptor, where a first portion of said receptor containing cells (“target cells”) differs from a second portion of said receptor containing cells (“non-target cells”) by either having a higher level of a surface membrane marker than said non-target cells or being in proximity to a first cell population having a surface membrane marker differing from a second cell population to which said non-target cells are in proximity, employing a conjugate of a binding member binding to said surface membrane marker conjugated to an attenuated ligand for said receptor, said attenuated ligand having a reduced physiological effect as compared to the wild-type ligand for said receptor, said method comprising:
combining said cells with said conjugate, whereby said conjugate binds to said surface membrane marker and said attenuated ligand binds to said receptor,
wherein the physiological function of said target cells is modulated and said non-target cells have substantially reduced physiological modulation.

2. A method according to claim 1, wherein said marker is a cancer cell marker.

3. A method according to claim 1, wherein said marker is a hematopoietic cell marker.

4. A method according to claim 1, wherein said marker has a naturally occurring protein ligand.

5. A method according to claim 1, wherein said marker is up regulated by an extracellular stimulus.

6. A method according to claim 1, wherein said marker is modified on said target cells as compared to said other cells.

7. A method for differentially modulating the activity of a predetermined receptor on target cells sharing the same predetermined receptor as non-target cells and differing by having a higher level of a surface membrane marker than said non-target cells, employing a conjugate of an antibody or binding fragment thereof binding to said surface membrane marker and conjugated to an attenuated protein ligand for said receptor, said attenuated ligand being less modulating than the wild-type ligand for said receptor, said method comprising:
combining said cells with said conjugate, whereby said conjugate binds to said surface membrane marker and said attenuated ligand binds to said receptor,
wherein said receptor activity on said target cells is modulated and the activity of said receptor on said non-target cells is substantially less.

8. A method according to claim 7, wherein said attenuated protein ligand is a mutated naturally occurring ligand.

9. A method according to claim 8, wherein said mutated protein ligand is a cytokine, chemokine or growth factor.

10. A method according to claim 7, wherein said antibody is IgG.

11. A method according to claim 7, wherein said binding fragment is scFv.

12. A method for differentially modulating the activity of a predetermined receptor on target cells sharing the same predetermined receptor as other cells (non-target cells), wherein said receptor containing cells (target cells) are in proximity to a mixed cell population comprising first and second cell populations and said target cells are distinguished from said non-target cells by being in proximity to a first cell population having a surface membrane marker substantially absent from a second cell population to which said second portion is in proximity, employing a conjugate of a binding member binding to said surface membrane marker conjugated to an attenuated ligand for said receptor, said attenuated ligand being less modulating than the wild-type ligand for said receptor, said method comprising:
combining said cells with said conjugate, whereby said conjugate binds to said surface membrane marker and said attenuated ligand binds to said receptor,
wherein said receptor activity on said target cells is modulated and modulation of said receptor activity on said non-target cells is substantially less.

13. A method according to claim 12, wherein said antibody is IgG.

14. A method according to claim 12, wherein said binding fragment is scFv.

15. A method according to claim 12, wherein said attenuated ligand is a modified naturally occurring molecule.

16. A method for differentially activating a predetermined receptor on target cells sharing the same predetermined receptor as non-target cells and wherein activation of said receptor results in a change in cell number, said target cells differing from said non-target cells by having a higher level of a surface membrane marker, employing a conjugate of a binding member binding to said surface membrane marker conjugated to an attenuated ligand for a receptor, said attenuated ligand being less activating than the wild-type ligand for said receptor and modulating cell number, said method comprising:
combining said cells with said conjugate, whereby said conjugate binds to said surface membrane marker and said attenuated ligand binds to said receptor,
wherein said receptor on said target cells is activated substantially greater than the activation of said receptor on said non-target cells.

17. A method according to claim 16, wherein said modulating cell number is inducing apoptosis.

18. A method according to claim 16, wherein said modulating cell number is inducing proliferation.

19. A method according to claim 16, wherein said modulating cell number is inhibiting proliferation.

20. A composition for use in the method of claim 1, comprising a conjugate of a binding member and an attenuated ligand, said binding member binding to a surface membrane marker on target cells sharing the same receptor as non-target cells and differing by having a higher level of said surface membrane marker, said attenuated ligand binding to said receptor inducing a physiological change in said target cells.
A composition according to claim 1, wherein said binding member is an antibody or binding fragment thereof and said ligand is a mutated naturally occurring protein.
